(19) United States
(12) Patent Application Publication
(10) Pub. No.: US 2006/0178677 A1
(43) Pub. Date: Aug. 10, 2006

(54) Hair punch

(75) Inventor: Gail L. Brinson, Brazoria, TX (US)

Correspondence Address: Gail L. Brinson;Technical Innovations, L.L.C., 20714 Highway 36, Brazoria, TX, 77422, US

(73) Assignee: Technical Innovations, L.L.C.

(21) Appl. No.: 11/054,876

(22) Filed: Feb. 10, 2005

(51) Int. Cl. A61B 17/50

(52) U.S. Cl. 606/133

(57) ABSTRACT

A hair punch is disclosed for use in transplantation of human hair follicles. The hair punch comprises a shank and an elongated tubular member that form an integral structure. The hair punch has proximal and distal ends. The distal end comprises a beveled cutting tip which is sharpened, and the proximal end is adapted to facilitate human manipulation of the hair punch. A lumen is formed in the hair punch between the proximal and distal ends. The lumen has a circular cross section along its length, and the lumen is uniformly tapered from a preselected point in the shank to the distal end. The elongated tubular member structure has a wall thickness that is substantially constant and less than 0.005 inches for a predetermined length of the elongated tubular member.

### BACKGROUND OF THE INVENTION

1. Field of the Invention

The present invention relates to a punch for use in hair transplantation surgical procedures.

2. Description of the Prior Art

For many individuals, hair loss can be undesirable. Many hair replacement alternatives have been proposed including hair transplant techniques. The hair transplant alternative is particularly attractive because living hair can be used to cover balding or bald areas. As the techniques for performing a hair transplantation have improved, this technique has become more widely accepted.

At the present time there are primarily two state of the art methods that are used to perform hair transplantation. One of these methods, and perhaps the most popular, is called a follicular unit transplantation or “strip method.” Using this technique, a strip of skin is removed from the back of the scalp. The area from which it was removed is then closed using sutures. The hair roots are then removed from the strip and are grafted into a number of incisions that have been made in the scalp. When using this technique, the average size of the strip may contain between about 1000 to 3000 grafts of hair follicles.

Another relatively new technique for hair transplantation is referred to as the follicular unit extraction or “FUE” technique. In using this technique, there is no need to remove a strip of skin or to use sutures. This procedure involves removing hair roots by first making an incision around the hair follicle using a sharpened punch, which penetrates generally from 1 to 4 millimeters into the scalp of the patient. Use of the punch weakens the structure around the hair follicle, which allows for removal of the hair follicle by using fine forceps. The removed hair follicles may then be grafted into incisions that have been made into the scalp to receive the hair follicles. With this technique there is usually no visible scaring and sutures are unnecessary.

The punch and forceps method described above was discussed and criticized in published patent applications US 2004/0116942 A1 and US 2004/0220589 A1. Each of these published applications criticizes the punch and forceps technique as being a technique where the follicular units are injured, crushed, torn or otherwise damaged. Each of these published patent applications discloses a perforation and suction technique for harvesting the follicular units.

It is believed that whatever problems may exist in using the punch and forceps technique are directly attributable to the geometry of the punches which have heretofore been available, and that such problems may be minimized, if not avoided, by employment of a hair punch with an improved geometry. That useful result has been achieved by the present invention.

### SUMMARY OF THE INVENTION

In accordance with the present invention, apparatus is provided for use in the transplantation of human hair follicles. The apparatus comprises a shank and an elongated tubular member which are formed as an integral structure. In one embodiment, the shank and elongated tubular member are a unitary structure and are fabricated from a single piece of stainless steel.

The apparatus has proximal and distal ends, and one end of the elongated tubular member comprises the distal end of the structure which is beveled and sharpened. In one embodiment the sharpened distal end is beveled at approximately 15 degrees. One end of the shank comprises the proximal end of the structure, and the shank is adapted for operative connection to a handle or other suitable device to facilitate human manipulation of the apparatus.

A central hole or lumen is formed in the apparatus between its proximal and distal ends. The lumen has a circular cross section along its length and the diameter of the lumen continually decreases from a preselected point in the shank to the distal end of the elongated tubular member.

The elongated tubular member has a wall thickness which is substantially constant for a predetermined length of the structure and which is less than 0.005 inch.

### BRIEF DESCRIPTION OF THE DRAWINGS

FIG. 1 is a perspective view of a hair punch in accordance with the present invention.

FIG. 2 is a cross-sectional view of the hair punch of FIG. 1 taken along a line 2-2′ in FIG. 1.

FIG. 3 is a perspective view of a handle for use with the hair punch of FIG. 1.

### DESCRIPTION OF SPECIFIC EMBODIMENTS

It will be appreciated that the present invention may take many forms and embodiments. Some embodiments of the invention are described so as to give an understanding of the invention. It is intended that the embodiments described herein should be illustrative, and not limiting of the invention.

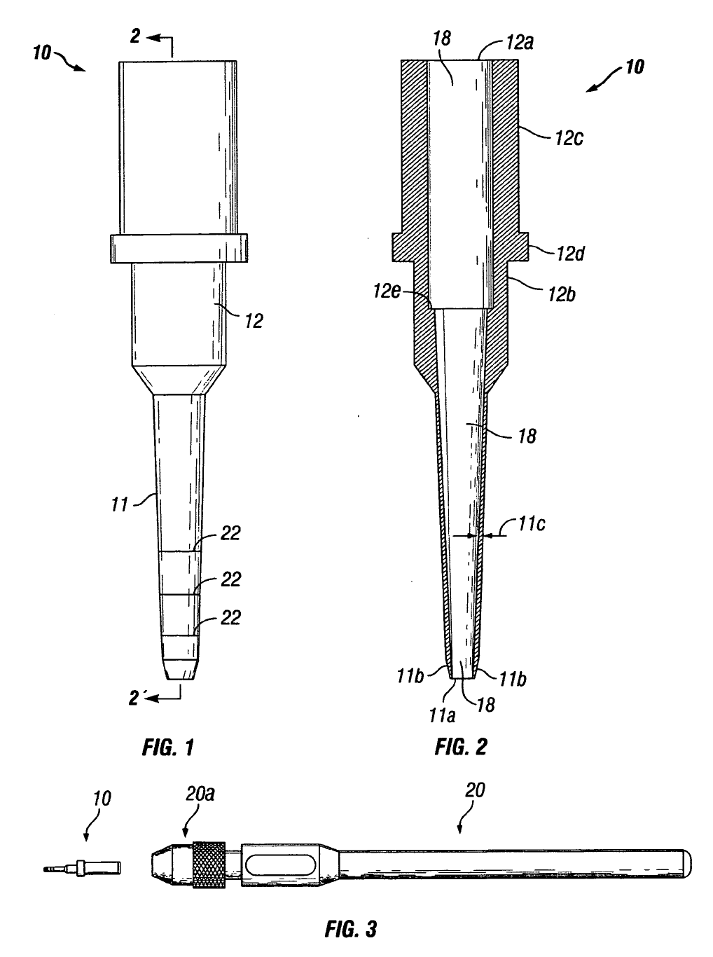

With reference first to FIGS. 1 and 2, there is illustrated a hair punch 10 in accordance with the present invention. Hair punch 10 comprises an elongated tubular member 11 and a shank portion 12 which are formed as an integral structure. The shank 12 may comprise a first portion 12b and a second portion 12c, where the diameter of the second portion 12c is greater than the diameter of the first portion 12b. The shank 12 may also comprise a flange 12d which is interposed between first shank portion 12b and second shank portion 12c. Shank portion 12 and elongated tubular member 11 may, for example, be fabricated as a unitary structure from a single piece of stainless steel.

With reference now to FIG. 2, the hair punch 10 in accordance with the present invention has a distal end and a proximal end. One end 11a of elongated tubular member 11 constitutes the distal end of hair punch 10, while one end 12a of shank 12 constitutes the proximal end of hair punch 10. The distal end 11a of hair punch apparatus 10 has a beveled cutting surface 11b which is sharpened. In one embodiment, the angle of the bevel for the cutting surface 11b is approximately 15°.

A central bore or lumen 18 is formed between the proximal end 12a and the distal end 11a of hair punch apparatus 10. Lumen 18 has a circular cross section at all points along its length. Further, the diameter of lumen 18 uniformly decreases or tapers from a preselected point 12e in the shank 12 to the distal end 11a of the elongated tubular member 11. In one embodiment, the aforesaid taper of lumen 18 is approximately 1.5° and the diameter of the hair punch at distal end 11a is approximately 1 mm.

Hair punch apparatus 10 in accordance with the present invention has an wall 11c having a thickness which is substantially constant and less than 0.005 inches for the length of the elongated tubular member 11 except for that portion of elongated tubular member 11 comprising the beveled cutting surface 11b. In one embodiment, the thickness of wall 11c is 0.003 inches.

Hair punch apparatus 10 in accordance with the present invention may further comprise markings 22 which are placed on the exterior of the elongated tubular member 11 to indicate the depth of insertion of the punch. These markings may, for example, be placed on the exterior of the elongated tubular member 11 by using well-known laser techniques or machining methods.

With reference to FIGS. 1 and 3, the shank 12 may advantageously be seated in the distal end 20a of an appropriate handle 20 to permit user manipulation of hair punch apparatus 10. In one embodiment, the handle 20 comprises a well-known pinvise device, and a flange 24 may be formed on shank 12 to prevent migration of hair punch 10 into the pinvise device during use.

It is believed that the design of a hair punch in accordance with the present invention has several advantages over prior art hair punches. First, the tapered lumen in a hair punch according to the present invention allows the surgeon to turn the hair punch once it is inserted in the patient's scalp with less risk of twisting and damaging the hair follicle being harvested. The tapered lumen is only in contact with the scalp tissue containing the hair follicle being harvested at the beveled cutting surface 11b, and the tapered lumen thus provides a relief area for the hair follicle being harvested. Second, some prior punches employ a radius cutting edge which tends to push scalp tissue aside, as opposed to cutting through the scalp tissue. A hair punch having a beveled cutting edge in conjunction with the thin outer wall 11c of elongated tubular member 11 encounters less resistance in moving through scalp tissue than do prior art punches. A punch in accordance with the present invention thus comprises a combination of geometries, both internal and external, that permit the punch to have substantial advantages over prior art punches.

What is claimed is:

1. A hair punch for use in the transplantation of human hair follicles, said hair punch having proximal and distal ends, comprising:
a shank having first and second ends, where the first end of the shank comprises the proximal end of the hair punch;
an elongated tubular member having first and second ends, where the first end of the elongated tubular member is attached to the second end of the shank, and the second end of the elongated tubular member comprises the distal end of the hair punch, said elongated tubular member having an wall thickness which is substantially constant for a predetermined length of the elongated tubular member; and
a lumen formed between the proximal and distal ends of the hair punch, the lumen having a circular cross section along its length and having a diameter which uniformly decreases from a preselected point in the shank to the proximal end of the hair punch.

2. The hair punch of claim 1, wherein the wall thickness of the elongated tubular member is less than 0.005 of an inch.

3. The hair punch of claim 1, wherein the distal end is beveled at an angle of about 15°.

4. The hair punch of claim 1, wherein the lumen is uniformly tapered from said preselected point in the shank to the distal end at a taper of approximately 1.5°.

5. The hair punch of claim 1, wherein the shank and elongated tubular member comprise a unitary structure and are fabricated from a single piece of stainless steel.
